(19) United States
(12) Patent Application Publication
(10) Pub. No.: US 2006/0105338 A1
(43) Pub. Date: May 18, 2006

(54) Method and apparatus for correcting position and attitude of object to be held

(76) Inventors: Akihiro Iimura, Tokyo (JP); Miki Tobita, Tokyo (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/525,751

(22) PCT Filed: Aug. 22, 2003
(86) PCT No.: PCT/JP03/10635

§ 371 Date: Sep. 22, 2005

(30) Foreign Application Priority Data
Aug. 29, 2002 (JP) 2002-250618
Aug. 20, 2003 (JP) 2003-296240

(51) Int. Cl. C12Q 1/68; G06F 19/00; C12M 1/34

(52) U.S. Cl. 435/6; 435/287.2; 702/20

(57) ABSTRACT

There is provided a method of correcting position and attitude of an object to be held capable of reducing the number of image processing steps and simplifying the method of correcting shifts in the position and attitude of the object with respect to a holding member. The object to be held is detachably held to the holding member and a fiducial mark 1 and a fiducial mark 2 are applied to the object. First, the fiducial mark 1 is subjected to an image processing so as to obtain position data of the fiducial mark 1 (S2). Next, the holding member holding the object to be held is rotated substantially by 180 degrees in a horizontal plane (S3). Then, the fiducial mark 2 rotated by 180 degrees is subjected to an image processing so as to obtain position data of the fiducial mark 2 (S4). Next, according to the position data of the fiducial mark 1 and the fiducial mark 2, a position shift amount from the rotational center of the holding member to the center of the object to be held is calculated and an angle shift amount of the holding member with respect to the fiducial line in the horizontal plane is calculated.

### FIELD OF THE INVENTION

The present invention relates to a method and an apparatus for correcting shifts, in position and attitude of an object to be detachably held to a holding portion, with respect to the holding position.

### BACKGROUND ART

Recently, a technology for effectively analysing entire gene functions of various organisms has been developed. A DNA micro-array (i.e., DNA tip) is an array in which a number of spots including DNA pieces are arranged on a substrate formed of slide glass or silicone, and is very effective for analysing expression, mutation (or variant) and diversity of genes.

A general substrate has a size or area of 1 to several tens cm², and in this area, several thousands to several hundred thousands kinds of spots of the DNA pieces are arranged. The DNA pieces on the substrate will be examined by using fluorescent labelling DNA having complementarity. When hybridization between the DVA pieces and the fluorescent labelling DNA is caused, fluorescence is generated. Spots generated by such fluorescence are detected by a fluorescent scanner or like to thereby analyse expression, mutation and diversity of the genes.

### DISCLOSURE OF THE INVENTION

In order to prepare a DNA micro-array, it becomes necessary to use an DNA micro-array preparing apparatus for arranging spots of closely densed DNA pieces on a substrate. A head, in which many kinds of DNA samples for forming the spots on the substrate are stored, is detachably held to the DNA micro-array preparing apparatus. The head after performing a spot forming working is dismounted from a holding portion and the next head, in which other DNA samples are stored, is held by the holding portion.

FIG. 9 shows a head 1 held to the holding portion. The head held by the holding portion is shifted in position and attitude from a preliminarily given fiducial position 1′. For this reason, in order to form a spot to an accurate position on the substrate, it is necessary to correct (or calibrate) a shift in position and angle of the exchanged head 1 with respect to the fiducial position 1′. This correction is performed, for example, as follows.

A fiducial mark 1 (FM1) and a fiducial mark 2 (FM2) are preliminarily applied to a position orthogonal to the head 1. The head 1 held to the holding portion is moved in the X-direction and Y-direction, and the fiducial mark 1 (FM1) is moved onto a CCD camera, at which a position data (x1, y1) of the fiducial mark 1 (FM1) is image-processed and then read out.

Thereafter, the head 1 is again moved in the X- and Y-directions, and the fiducial mark 2 (FM2) is moved onto the CCD camera, at which a position data (x2, y2) of the fiducial mark 2 (FM2) is image-processed and then read out.

A shifted angle θ of the head 1 in a horizontal plane with respect to a fiducial line (i.e., a line connecting the fiducial mark 1 (FM1′) and the fiducial mark 2 (FM2′) at the fiducial position 1′) of the holding portion is operated from such position date as shown in FIG. 9.

Next, as shown in FIG. 10, the head 1 is rotated in the horizontal plane by an angle corresponding to the shifted (shift) angle θ so as to take a position of the head 1 to be parallel the fiducial position 1′. The position data of the fiducial mark 1 (FM1) and the fiducial mark (FM2) of the head 1 are again processed and read out to thereby operate a shifted (shift) amount (xo, yo) of the center O of the head with respect to a rotation center O′ of the holding portion.

The shift amount (xo, yo) of the center O of the head 1 and the shift angle amount θ are measured every time of changing the head, and in accordance with the measured value, the shifting in position and attitude of the heat 1 at the time of forming the spots on the substrate can be corrected.

In the correction method mentioned above, however, four times of image-processing workings, in total, are required for measuring the shift amount of the center of the head and the angle shift amount thereof, which needs a troublesome correction working, thus providing a problem.

Taking the above matters into consideration, the present invention aims to provide a method and an apparatus for correcting position and attitude of an object to be held by a holding portion capable of reducing the number of image processing working and simplifying a method of correcting shifts in position and attitude of the object to be held with respect to the holding portion.

Means for Solving the Problems

Hereunder, the present invention will be explained. In order to achieve the above object, the inventors of this application read out a fiducial mark 2 rotated by 180 degrees by rotating the object to be held substantially by 180 degrees in a horizontal plane after reading out the fiducial mark 1 of the object.

That is, the above object is achieved by providing, as an invention of claim 1, a method of correcting shifts in position and attitude of an object which is held by a holding member and to which fiducial mark 1 and fiducial mark 2 are applied, the method comprising the steps of: obtaining a position data of the fiducial mark 1 by image-processing the fiducial mark 1; rotating the holding member holding the object to be held substantially by 180 degrees in a horizontal plane; obtaining a position data of the fiducial mark 2 by image-processing the fiducial mark 2 rotated by 180 degrees; and operating, on the basis of the position data of the fiducial mark 1 and the fiducial mark 2 rotated by 180 degrees, an amount of position shift from a rotational center of the holding member to a center of the object to be held and an amount of angle shift of the object in a horizontal plane with respect to a fiducial line of the holding member.

According to this invention, the positional shift amount and the angular shift amount of the center of the holding member can be obtained by two times of image processing.

Furthermore, according to the present invention, there is also provided a program for correcting, by using a computer, shifts in position and attitude of an object which is held by a holding member and to which fiducial mark 1 and fiducial mark 2 are applied, the program comprising: a sequence for obtaining a position data of the fiducial mark 1; a sequence for rotating the holding member holding the object to be held substantially by 180 degrees in a horizontal plane; a sequence for obtaining a position data of the fiducial mark 2 rotated by 180 degrees; and a sequence for executing an operation, on the basis of the position data of the fiducial mark 1 and the fiducial mark 2 rotated by 180 degrees, for calculating an amount of position shift from a rotational center of the holding member to a center of the object to be held and calculating an amount of angle shift of the object in a horizontal plane with respect to a fiducial line of the holding member.

Still furthermore, the present invention also provides an apparatus for correcting shifts in position and attitude of an object which is held by a holding member and to which fiducial mark 1 and fiducial mark 2 are applied, the apparatus comprising: an imaging device for imaging the fiducial mark 1 and fiducial mark 2; an image-processing device for processing image information obtained by the imaging device to thereby obtain a position data; a rotating mechanism for rotating the holding member holding the object to be held substantially by 180 degrees in a horizontal plane; and an operation unit for operating, on the basis of the position data of the fiducial mark 1 and the fiducial mark 2 rotated by 180 degrees, an amount of position shift from a rotational center of the holding member to a center of the object to be held and an amount of angle shift of the object in a horizontal plane with respect to a fiducial line of the holding member.

As mentioned above, according to the present invention, since the fiducial mark 2 rotated by 180 degrees by rotating the object to be held substantially by 180 degrees in a horizontal plane after reading out the fiducial mark 1 of the object, the positional shift amount and the angular shift amount of the center of the holding member can be obtained by two times of image processing.

### BRIEF DESCRIPTION OF THE DRAWINGS

FIG. 1 is a side view of a DNA micro-array preparing apparatus.

FIG. 2 is a sectional view taken along the line II-II in FIG. 1.

FIG. 3 is a perspective view of a head.

FIG. 4 is a bottom view of the head.

FIG. 5 is a diagram of a control system of the DNA micro-array preparing apparatus.

FIG. 6 is a flowchart representing sequences executed by a computer.

FIG. 7 is an illustration of a mode showing shifts in position and angle of the head.

FIG. 8 is a geometrical view for operating a shift position amount and a shift angle amount.

FIG. 9 is an illustration showing a mode of shift in position in a head of conventional structure.

FIG. 10 is an illustration of a mode showing shifts in position and angle of the conventional head when it is minutely rotated.

### BEST MODE FOR EMBODYING THE INVENTION

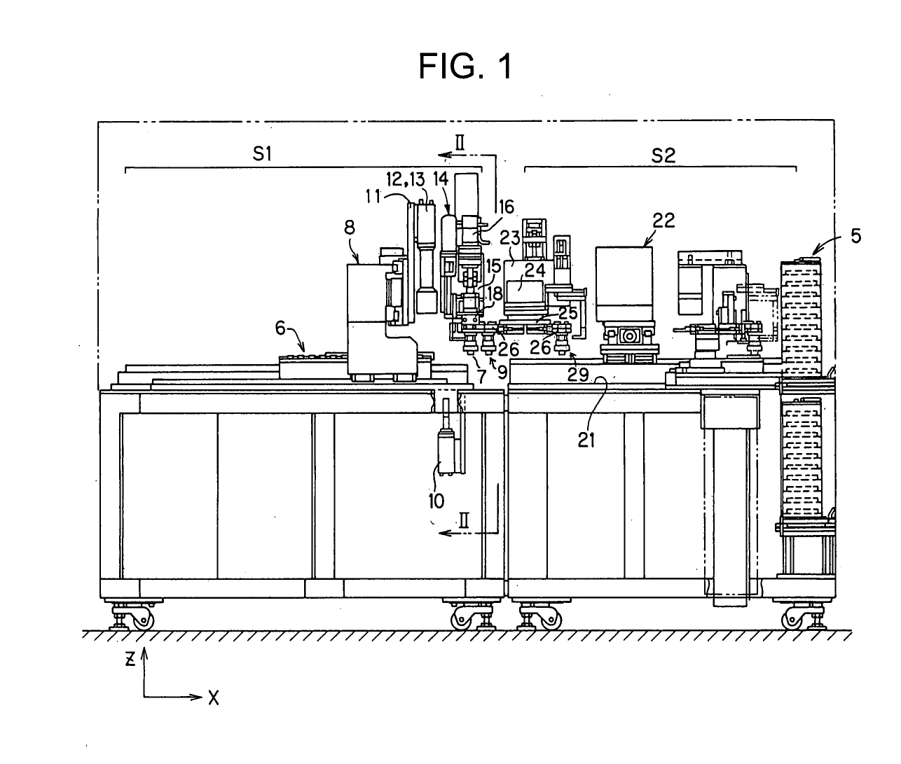

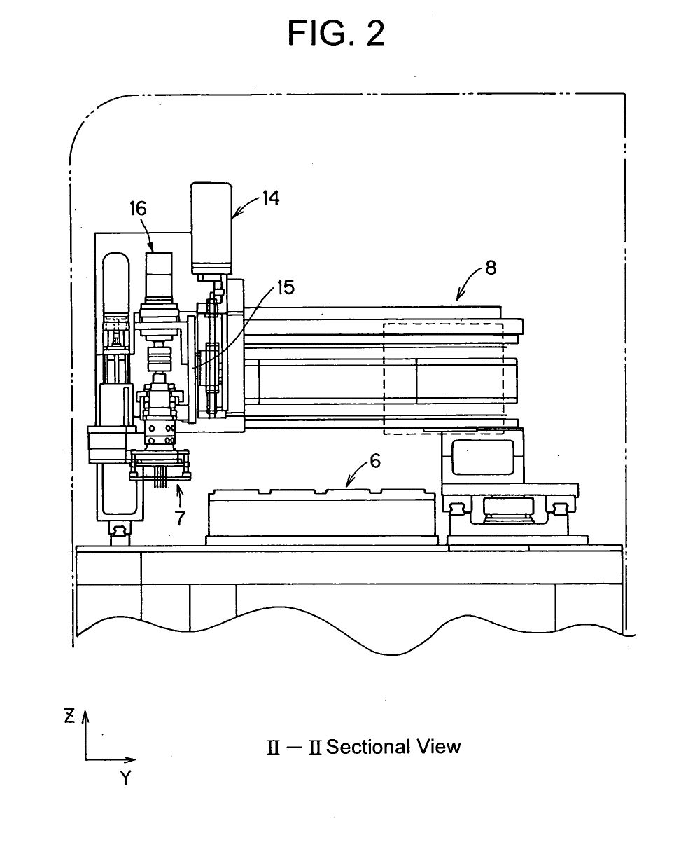

One embodiment of the present invention will be described hereunder with fiducial to the accompanying drawings. FIG. 1 is a side view of a DNA micro-array preparing apparatus as a position correction apparatus, and FIG. 2 is a sectional view of the apparatus as viewed from the direction of II-II line in FIG. 1. The DNA micro-array preparing apparatus of this embodiment is an apparatus for correcting a position of a head as an object to be held.

The DNA micro-array preparing apparatus is a apparatus for arranging spots of preliminarily prepared biological sample, such as DNA pieces or oligonucleotide, on a substrate formed of slide glass, silicone or like, and a solution containing the biological sample is stored in a solution storage container 5. A plurality of substrates applied with fiducial marks in form of vertical and horizontal matrix along the same plane are arranged on a working table 6. Spots of the solution are formed on the substrates by a head 7 arranged above the substrates to be movable. A general substrate has a size of 1 to several tens cm², and spots of several thousands to several hundred thousands of DNA pieces are arranged in form of vertical and horizontal matrix. Each of the spots has a diameter of, for example, several tens to several hundreds μm.

The DNA micro-array preparing apparatus has two areas or sections, one being a stamping area S1 for stamping the solution of the biological sample against the substrate holding the head 7 so as to form and arrange the spots of the solution onto the substrate, and the other one being a cleaning area S2 for cleaning the head after the formation of the spots and holding the next solution of different kind to the cleaned head 7. The head 7 is conveyed from the stamping area S1 and cleaning area S2 by conveying devices provided for the respective areas S1 and S2.

Further, a structure or configuration of the DNA micro-array preparing apparatus in the stamping area S1 is first described schematically hereunder. A plurality of substrates are placed on the working table 6 in the stamping area S1 in form of matrix. Each of the substrates is formed of a slide glass or silicone, and a pattern for forming fiducial marks and spots is formed on the surface of the substrate by a lithograph.

A biaxial (X-Y two-axes) conveyance mechanism 8 for moving the head 7 in two (X and Y) axial directions perpendicular to each other in a plane parallel to the substrate is mounted to the working table 6. This X-Y biaxial conveyance mechanism 8 operates so as to position the head 7 to the spot forming position on the substrate. Moreover, this X-Y biaxial conveyance mechanism 8 is moved to a head receiving position 9, which will be described hereinafter, to receive the head 7 holding new solution and then moved so as to convey the received head 7 to an imaging position on a head imaging device 10.

In addition, the X-Y biaxial conveyance mechanism 8 is provided with a mount table 11 to which a substrate imaging device (for example, CCD camera) 12 for imaging the fiducial mark on the substrate and a spot imaging device (for example, CCD camera) 13 for imaging the spot formed on the substrate are mounted.

Furthermore, a Z-axis driving mechanism 14 is supported by the mount table 11, and this Z-axis driving mechanism 14 operates so as to move the head 7 in the Z-axis direction perpendicular to the X-axis and Y-axis directions, that is, in directions approaching to or separating from the substrate.

The Z-axis driving mechanism 8 is provided with a table 15 to which a θ-axis rotation mechanism 16 for changing the attitude of the head 7, and this θ-axis rotation mechanism 16 acts to turn the head 7 in the horizontal plane. A holding member 18 for detachably holding the head 7 is mounted to the θ-axis rotation mechanism 16. When the θ-axis rotation mechanism 16 is driven, the head 7 is rotated around the Z-axis, so that the attitude of the head 7 changes. In addition, when the X-Y biaxial conveyance mechanism 8 is operated, the position of the head 7 changes.

As shown in FIG. 1, the head imaging device (for example, CCD camera) 10 is mounted to the working table 6 for imaging the attitude and position of the head 7 from the lower side thereof. The newly exchanged head 7 is first conveyed above this head imaging device 10, and although described in detail hereinafter, the fiducial mark 1 and fiducial mark 2 for indicating the position of the head 7 are marked to the lower surface of the head 7, and the head imaging device 10 images the fiducial marks 1 and 2.

Next, the structure or configuration of the DNA micro-array preparing apparatus in the cleaning area S2 will be described. In this cleaning area S2, the head 7 after forming the spots is cleaned by means of ultrasonic cleaning, rinsed and then dried. After the cleaning, solution of the new next biological specimen is stored to the cleaned head 7.

As shown in FIG. 1, an X-Y biaxial conveyance mechanism 22 for conveying the head 7 between an ultrasonic cleaning section, a rinse-cleaning section, a drying section and a solution storage section is mounted on a cleaning table 21 in the cleaning area S2. A Z-axis driving mechanism is also mounted to the X-Y biaxial conveyance mechanism 22. The Z-axis driving mechanism is driven so as to move the head 7 in a Z-axis direction normal to the X-axis and Y-axis direction, i.e., direction perpendicular to the cleaning table 21.

The Z-axis driving mechanism includes a table 23 to which a motor 24 for turning is secured, and a disc 25 turning in the horizontal plane is mounted to an output shaft of the turning motor 24. A pair of clamps 26, 26 capable of holding the head 7 are provided for the lower surface of the disc 25 at an angular interval of 180 degrees. These clamps 26, 26 are operated to be opened or closed by an air cylinder, not shown, so as to clamp the head 7.

The turning motor 24 turns by every 180 degrees to thereby carry out the transfer, i.e., delivery and receipt, of the head 7 from the X-Y biaxial conveyance mechanism 8 in the stamping area S1 to the X-Y biaxial conveyance mechanism 22 in the cleaning area S2 and the transfer, i.e., delivery and receipt, of the head 7 from the X-Y biaxial conveyance mechanism 22 in the cleaning area S2 to the X-Y biaxial conveyance mechanism 8 in the stamping area S1.

More specifically, the X-Y biaxial conveyance mechanism 8 in the stamping area S1 conveys the head 7, after being formed with the spots, to the transferring portion 9. On the other hand, the X-Y biaxial conveyance mechanism 22 in the cleaning area S2 conveys the head 7 holding the new solution from the head transferring position 9 to a waiting position 29 which is displaced by 180 degrees from the transferring position 9. In the next process, the clamps 26, 26 of the X-Y biaxial conveyance mechanism 22 in the cleaning area S2 clamp the head 7 conveyed, after being formed with the spots, to the transferring position 9, thereby transferring the head 7 from the X-Y biaxial conveyance mechanism 8 in the stamping area S1 to the X-Y conveyance mechanism 22 in the cleaning area S2. Next, the turning motor 24 is driven to turn the disc 25 by 180 degrees so that the head 7 after forming the spots is moved to the waiting position 29 and the head 7 holding the new solution is moved to the transferring position 9. In the next step, the holding member 18 of the X-Y biaxial conveyance mechanism 8 of the stamping area S1 holds the head 7 holding the new solution, thereby the head 7 is transferred from the X-Y biaxial conveyance mechanism 22 in the cleaning area S2 to the X-Y biaxial conveyance mechanism 8 in the stamping area S1.

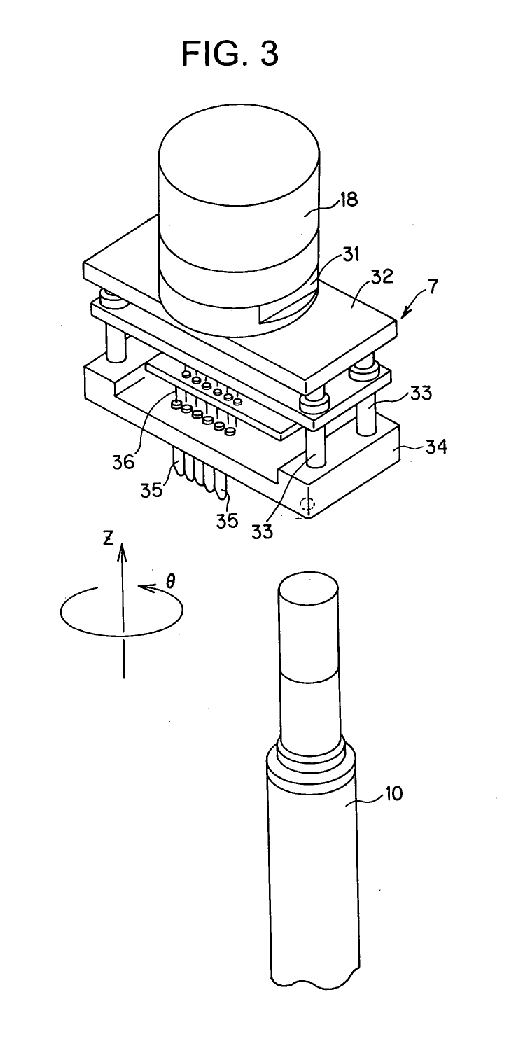

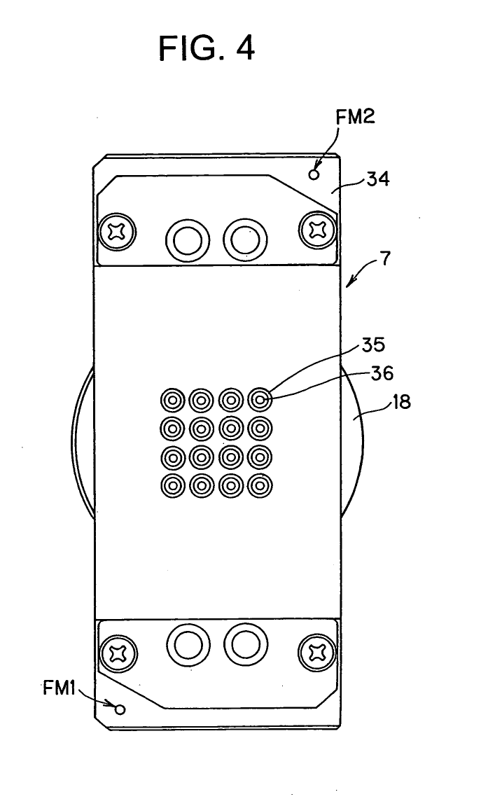

FIGS. 3 and 4 represents the head 7. The head 7 comprises a cylindrical portion 31 to be held, which is secured to the holding member 18, an upper plate 32 having an approximately rectangular shape, which is fixed to the lower surface of the portion 31 to be held, and a lower plate 34 having an approximately rectangular shape, which is connected to the upper plate 32 through a plurality of support columns 33.

A number of solution holding members 35 - - - , serving as solution storage section, holding the solution to be supplied to the substrate, are disposed to the lower plate 34 in an arrangement of vertical and horizontal rows in parallel to each other. Needles or pins 36 - - - are accommodated in the solution storage section 35. These needles 36 - - - are arranged so as to project over the solution storage section 35, and the front ends thereof are beaten against the substrate, thereby distributing the solution adhering to the front ends of the needles 36 - - - to the substrate.

Further, various systems for arranging the solution to the substrate may be adopted other than the above system including the structure of solution storage section for holding the solution and the arranging section (for example, pins or needles) for taking out the solution from the solution storage section and arranging the solution as spots by mechanically abutting against the substrate, the other various systems including: a pen system in which a specimen is held in an opened capillary passage formed between a pair of members, like pens, disposed with a space from each other, and the front ends of the paired fine members mechanically abut against the substrate; an inkjet system utilizing a theory of an inkjet printer; and a capillary system utilizing a capillary tube.

FIG. 4 is a bottomed view of the head 7. Two fiducial marks 1 and 2 are applied to the diagonal positions on the bottom surface of the rectangular lower plate 34 of the head 7. These fiducial mark 1 (FM1) and fiducial mark 2 (FM 2) are formed as circular holes, respectively.

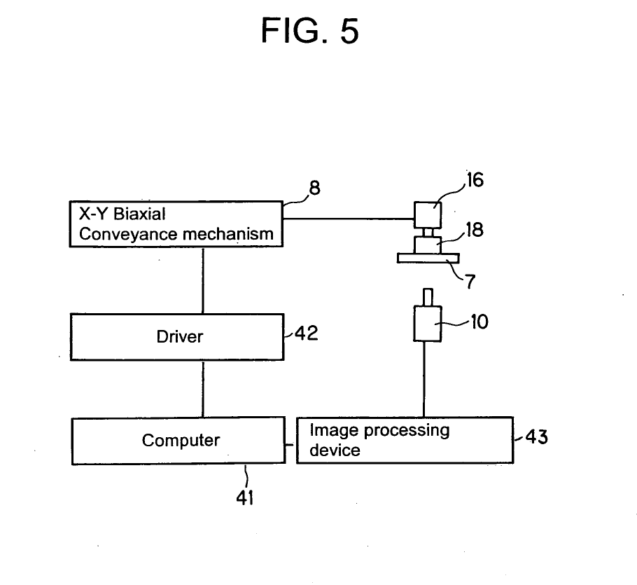

FIG. 5 shows a system diagram of a control system of the DNA micro-array preparing apparatus of the structure mentioned above, and herein, a control system for correcting the shifts in position and attitude of the head 7 with respect to the holding member 18 will be explained. This control system is provided with a computer, such as personal computer, 41 totally controlling the operation of the DNA micro-array preparing apparatus in accordance with a predetermined program, a head imaging device 10 arranged on a mechanically base point, an image processing device 43 for processing information of the image imaged by the head imaging device 10, and a driver 42 for controlling the driving operations of the X-Y biaxial conveyance mechanism 8 in the stamping area S1 and the θ-axis rotation mechanism 16 in response to commands from the computer 41. The image processing device 43 calculates the position data of the fiducial marks FM1 and FM 2 of the head 7 based on the image information from the head imaging device 10 and outputs the thus calculated position data to the computer 41.

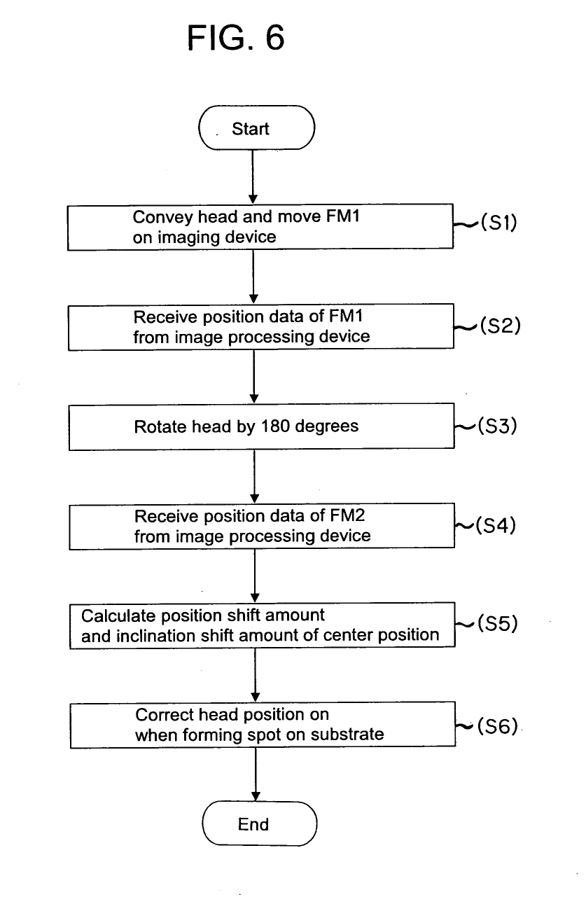

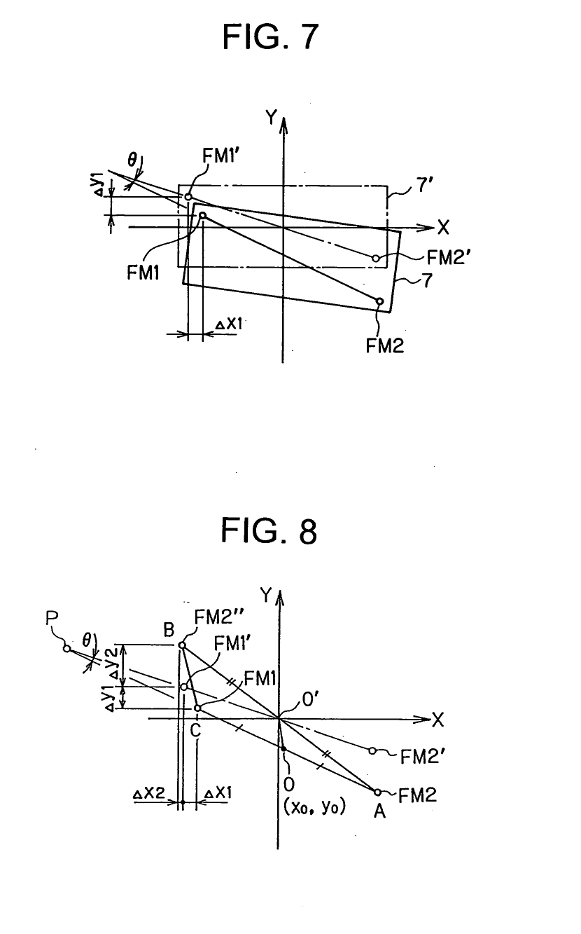

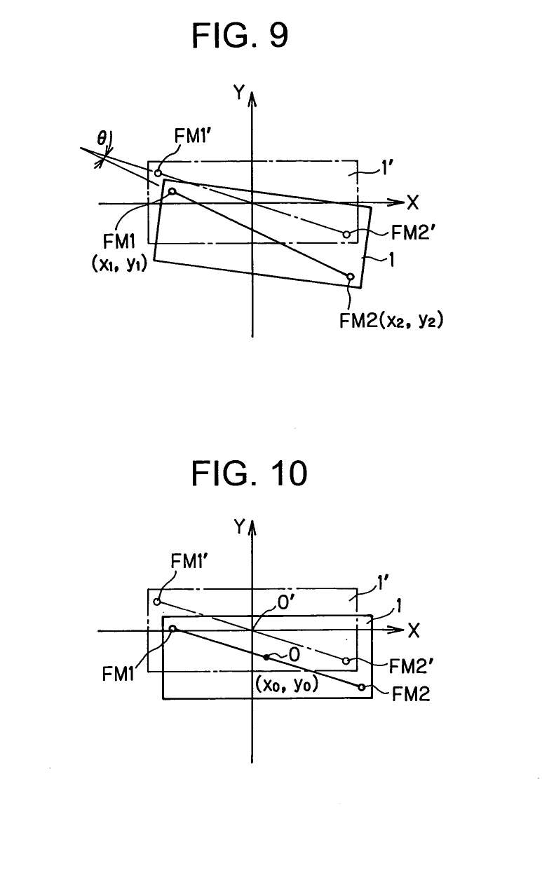

FIG. 6 shows a flowchart representing the sequence, executed by the computer 41, for correcting the position shift and attitude shift of the head 7 with respect to the holding member 18. As shown in FIG. 7, the head held by the holding member 18 is displaced (shifted) in the position and attitude from the fiducial position 7′.

As shown in FIG. 6, the head 7 held by the holding member 18 is first conveyed, and the fiducial mark 1 (FM1) marked to the head 7 is moved onto the head imaging device 10 (step S1). Next, the image processing unit 43 processes the image of the fiducial mark 1 (FM1) so as to obtain the position data of the fiducial mark 1 (FM1). For example, this position data is obtained as position (positional) shift amount (Δx1, Δy1) displaced from the fiducial mark 1′ (FM1′) of the fiducial position 7′. The computer 41 reads in this position data of the fiducial mark 1 (FM1) calculated by the image processing unit 43 (step S2).

In the next step, the holding member 18 holding the head 7 is rotated substantially by 180 degrees in the horizontal plane without moving in the X-axis and Y-axis directions (step S3). At this moment, the fiducial mark 2 (FM2) is moved, as shown in FIG. 8, to a position FM2″ which is point-symmetric with the rotational center O′ of the holding member 18. At this time, the image processing unit 43 processes the image of the fiducial mark 2 (FM2) so as to obtain the position data of the fiducial mark 2 (FM2″) which is rotated by 180 degrees. For example, this position data is obtained as position shift amount (Δx2, Δy2) displaced from the fiducial mark 1′ (FM1′) of the fiducial position 7′. The computer 41 reads in this position data of the fiducial mark 2 (FM2″) calculated by the image processing unit 43 (step S4).

Next, on the basis of the position data of the fiducial mark 1 (FM1) and the above-mentioned fiducial mark 2 (FM2″) which is rotated by 180 degrees, the position shift amount from the rotational center O′ of the holding member 18 to the center of the head 7 and the angle (angular) shift amount of the object to be held, in the horizontal plane, with respect to the fiducial line of the holding member are operated (step S5).

More specifically, the position shift amount is obtained in the following manner. As shown in FIG. 8, the triangle ABC and the triangle AO′O are in analogous relation (similar shape), and accordingly, when it is supposed that the rotational center O′ of the holding member 18 accords with the origin of the coordinate, the coordinate of the center O of the head 7 will be given as 1/2(Δx1+Δx2, Δy1+, Δy2).

Further, the inclination angle θ of the head 7 will be obtained from the position data of the fiducial mark 1 (FM1) of the head 7, the position data of the fiducial mark 1 (FM1′) at the fiducial position 7′ of the head 7, and the position shift amount of the center O of the head 7.

Then, an intersection point P of a line passing the fiducial mark 1 (FM1′) at the fiducial position 7′ and the rotational center O′ of the holding member 18 and a line passing the fiducial mark 1 (FM1) of the head 7 and the center O of the head 7 is obtained, in which:
\vec{PO′}=\overline{PO′}ₑ^{j θ}^{^{PO′}}, \vec{PO}=\overline{PO}ₑ^{jθ}^{^{PO}} [Equation 1]

The inclination angle θ of the head 7 is obtained as follows by setting the vector shown in the above Equation 1 with the point P being the fiducial point.
θ=θ^{PO}−θ^{PO′}

The computer 41 stores, in its memory section, the position shift amount and the inclination angle amount and then operates the X-Y biaxial conveyance mechanism 8 and the 0 axis rotation mechanism 16 in the stamping area S1 via the driver so as to correct the position of the head in accordance with these position shift amount and the inclination angle amount.

Further, it is to be noted that the present invention is not limited to the embodiment described above and many other changes and modifications may be made without departing from the subject matters of the present invention. For example, an object to be held by the holding member is not limited to the head for the DNA micro-array preparing apparatus as far as it can be detachable to the holding member, and electronic parts or likes equipped to a printed circuit board or like may be utilized. In addition, the calculation of the position shift amount and inclination angle amount may be done by methods other than the calculation method described in the above disclosure, and other geometrical means may be utilized.

1. A method of correcting shifts in position and attitude of an object which is held by a holding member and to which fiducial mark 1 and fiducial mark 2 are applied, said method comprising the steps of:
obtaining a position data of the fiducial mark 1 by image-processing the fiducial mark 1;
rotating the holding member holding the object to be held substantially by 180 degrees in a horizontal plane;
obtaining a position data of the fiducial mark 2 by image-processing the fiducial mark 2 rotated by 180 degrees; and
operating, on the basis of the position data of the fiducial mark 1 and the fiducial mark 2 rotated by 180 degrees, an amount of position shift from a rotational center of the holding member to a center of the object to be held and an amount of angle shift of the object in a horizontal plane with respect to a fiducial line of the holding member.

2. The method of correcting shifts in position and attitude of an object according to claim 1, wherein the object to be held is a head of a DNA micro-array preparing apparatus for arranging a number of spots on a substrate.

3. A program for correcting, by using a computer, shifts in position and attitude of an object which is held by a holding member and to which fiducial mark 1 and fiducial mark 2 are applied, said program comprising:
a sequence for obtaining a position data of the fiducial mark 1
a sequence for rotating the holding member holding the object to be held substantially by 180 degrees in a horizontal plane;
a sequence for obtaining a position data of the fiducial mark 2 rotated by 180 degrees; and
a sequence for executing an operation, on the basis of the position data of the fiducial mark 1 and the fiducial mark 2 rotated by 180 degrees, for calculating an amount of position shift from a rotational center of the holding member to a center of the object to be held and calculating an amount of angle shift of the object in a horizontal plane with respect to a fiducial line of the holding member.

4. An apparatus for correcting shifts in position and attitude of an object which is held by a holding member and to which fiducial mark 1 and fiducial mark 2 are applied, said apparatus comprising:
an imaging device for imaging the fiducial mark 1 and fiducial mark 2;
an image-processing device for processing image information imaged by the imaging device to thereby obtain a position data;
a rotating mechanism for rotating the holding member holding the object to be held substantially by 180 degrees in a horizontal plane; and
an operation unit for operating, on the basis of the position data of the fiducial mark 1 and the fiducial mark 2 rotated by 180 degrees, an amount of position shift from a rotational center of the holding member to a center of the object to be held and an amount of angle shift of the object in a horizontal plane with respect to a fiducial line of the holding member.
